(19) United States
(12) Patent Application Publication
(10) Pub. No.: US 2006/0241031 A1
(43) Pub. Date: Oct. 26, 2006

(54) Assay

(76) Inventors: Per Akesson, Lund (SE); Lars Björck, Lund (SE); Anders Sjöholm, Lund (SE)

Correspondence Address: FOLEY AND LARDNER LLP;SUITE 500, 3000 K STREET NW, WASHINGTON, DC, 20007, US

(21) Appl. No.: 10/545,904

(22) PCT Filed: Feb. 19, 2004
(86) PCT No.: PCT/EP04/01581

§ 371 Date: Nov. 22, 2005

(30) Foreign Application Priority Data
Feb. 19, 2003 (GB) 0303809.8

(51) Int. Cl. A61K 38/16; G01N 33/554; C12Q 1/18

(52) U.S. Cl. 514/12; 435/32; 435/7.32

(57) ABSTRACT

An assay method for an anti-bacterial agent comprising: (a) providing as a first component protein SIC; (b) providing as a second component an antibacterial peptide; (c) contacting the first component with a test substance in the presence of the second component; and (d) determining the interaction or activity of the first component with the second component to determine thereby whether a test substance is an effective anti-bacterial agent.

### FIELD OF THE INVENTION

The invention relates to assays for antibacterial agents, and in particular antibacterial agents useful in the treatment of streptococcal infections.

### BACKGROUND TO THE INVENTION

S. pyogenes is one of the most common and important human bacterial pathogens. It causes relatively mild infections such as pharyngitis (strep throat) and impetigo, but also serious clinical conditions like rheumatic fever, post-streptococcal glomerulonephritis, necrotizing fasciitis, septicemia, and a toxic-shock syndrome. Increases in the number of life-threatening systemic S. pyogenes infections have been reported world-wide since the late 1980's, and have attracted considerable attention and concern. Based on the highly polymorphic M protein, a surface protein of S. pyogenes, isolates are divided into more than 100 serological subtypes and systemic infections are most frequently caused by organisms of the M1 serotype.

Protein SIC was originally isolated from the growth medium of an M1 strain (1). All strains of the M1 serotype secrete SIC and so do M57 organisms, whereas strains of 53 other serotypes were found to lack the sic gene (1). Subsequent work has identified sic homologues also in M12 and M55 strains (1). The name SIC stands for streptococcal inhibitor of complement, as the protein incorporates into the membrane attack complex (MAC) of complement and inhibits complement-mediated lysis of sensitized erythrocytes (1). This inhibition of MAC was recently shown to be the result of SIC preventing uptake of C567 onto cell membranes (3). A remarkable property of SIC was reported by Stockbauer et al. (4). They found that the sequences of a large number of sic genes from different strains of the M1 serotype, showed a unique degree of variation, which is in striking contrast to the lack of M1 protein variation. Moreover, in a mouse model of infection, Hoe et al. (5) discovered that SIC variants arise rapidly on mucosal surfaces by natural selection. They also reported that the inhibition of complement-mediated lysis by SIC, was not affected in the new SIC variants arising from natural selection, suggesting that complement inhibition is not the only function of SIC.

Complement belongs to the innate immune system and antibacterial peptides represent another import ant part of this defence system. These peptides, originally described in silk worms, play important roles in the clearance of bacteria at biological boundaries susceptible for infection (6-9).

### SUMMARY OF THE INVENTION

In accordance with the present invention, there is provided an assay method for an anti-bacterial agent comprising:

(a) providing as a first component protein SIC;

(b) providing as a second component an antibacterial peptide;

(c) contacting the first component with a test substance in the presence of the second component; and

(d) determining the interaction or activity of the first component with the second component to determine thereby whether a test substance is an effective anti-bacterial agent.

Inhibitors of protein SIC, for example identified in accordance with the present invention may be used in the treatment of prophylaxis of S. pyogenes infection. The protein SIC inhibitors may be used together with antibacterial peptides to treat S. pyogenes infection.

### DESCRIPTION OF THE FIGURES

FIG. 1. Sic interacts with antibacterial peptides. (A) Various amounts of the antibacterial peptides α-defensin and LL-37, and the peptide GCP derived from human H-kininogen, were applied to a PVDF membrane. The membrane was incubated with radiolabelled protein SIC (2×10⁵ cpm/ml) for 3 h and autoradiographed for 3 days. (B) Microtiter plates were coated with protein SIC or M1 protein at 2.9 nM, followed by incubation with α-defensin or LL-37 (58 nM). Binding was detected with specific antibodies against α-defensin and LL-37, respectively. The bars represent the mean±SEM of at least three experiments.

FIG. 2. SIC protects S. pyogenes against antibacterial peptides. AP1 bacteria (2×10⁶ cfu/ml) were incubated with the antibacterial peptides α-defensin (◯) or LL-37 (Δ) at indicated concentrations for 2 h at 37° C. and cfus were determined (left panel). The bactericidal effect of α-defensin (◯) or LL-37 (Δ), at a concentration of 448 nM, was inhibited with, various concentrations of protein SIC (right panel). Experiments were repeated at least three times and representative experiments are shown.

FIG. 3. Different SIC homologues block the bactericidal effect of α-defensin and LL-37. (A) Different homologues of protein SIC (1 μg) were subjected to SDS-PAGE (10% gel) and stained with Coomassie blue. Protein SICM1 is purified from S. pyogenes strain AP1; protein SICM12 from S. pyogenes strain AP12; and protein SICM55 from S. pyogenes strain W38. (B) Microtiter plates were coated with the different homologues of protein SIC shown in panel A, or with M1, protein, at 2.9 nM, followed by incubation with α-defensin or LL-37 (58 nM). Binding was detected with specific antibodies against α-defensin and LL-37, respectively. Lane 1: M1 protein; Lane 2: protein SICM1; Lane 3: protein SICM12; Lane 4: protein SICM55. The bars represent the mean±SEM of at least three experiments. (C) AP1 bacteria were incubated with antibacterial peptides (448 nM) for 2 h (α-defensin) or 1 h (LL-37) in the presence of various amounts of proteins SICM1 (◯), SICM12. (□), SICM55 (Δ), or M1 protein (⋄). The different preparations of protein SIC shown in panel A were used as inhibitors. Experiments were repeated at least three times and representative experiments are shown.

### DETAILED DESCRIPTION OF THE INVENTION

We have shown that protein SIC plays a role in inactivating anti-microbial peptides. The ability to inactivate these anti-microbial peptides increases the virulence of bacterial infection. This activity presents a novel target for the identification of antibacterial agents, in particular which can be used to inhibit the activity of protein SIC and therefore allow endogenous or administered antibacterial peptides to maintain their activity.

The invention provides methods for identifying an anti-bacterial agent. A suitable method of the invention comprises (a) providing as a first component, protein SIC; (b) providing as a second component an antibacterial peptide; (c) contacting the two components with a test substance; and (d) determining whether the test substance is capable of modulating the interaction between protein SIC and the antibacterial peptide.

Protein SIC or a functional variant thereof is provided as one component for use in the assays of the invention. Protein SIC can be provided from any suitable source. The amino acid sequence of protein SIC from the M1 strain of S. pyogenes is set out in SEQ ID NO: 1. Any suitable protein SIC can be provided including protein SIC derived from M1 serotype, M57 serotype, M12 serotype and M55 serotype or any other serotypes of S. pyogenes which expresses protein SIC. Examples of SIC genes and the encoded proteins are described in Stockbauer et al (4) and are suitable proteins for use in the methods of the present invention.

A functional variant of protein SIC has a sequence similar to that of SEQ ID NO: 1 and retains the ability to interact with and/or to interfere with the activity of antibacterial peptides. Typically, the activity of a functional variant of SIC is substantially the same as that wild type protein SIC. Alternatively, the activity may be greater or less than that of protein SIC. For example, a functional variant may have at least 90% activity, at least 80% activity or at least 70% activity of protein SIC of SEQ ID NO: 1 with respect to its ability to interact with and/or inactivate antibacterial peptides.

A functional variant typically comprises a sequence similar to that set out in the amino acid sequence of SEQ ID NO: 1.

Thus a functional variant will generally have at least 60%, at least 70%, at least 80%, at least 90%, at least 95%, at least 98 or at least 99% sequence identity to the protein SIC having the sequence set out as the amino acid sequence of SEQ ID NO: 1, calculated over the full length of those sequences. In the alternative, a finctional variant will generally have at least 60%, at least 70%, at least 80%, at least 90%, at least 95%, at least 98% or at least 99% sequence identity to protein SIC derived from another strain of S. pyogenes. The UWGCG Package provides the BESTFIT program which can be used to calculate identity (for example used on its default settings) (Devereux et al (1984) Nucleic Acids Research 12, p 387-395). The PILEUP and BLAST algorithms can be used to calculate identity or line up sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Centre for Biotechnology Information (http://www.ncbi.nlm.nih.gov/).

A functional variant may be a naturally occurring sequence, such as a related gene from another strain of the M1 serotype, or a variant from another strain of a different serotype such as a strain of the M12, M55 or M57 serotype.

Alternatively, a functional variant may be a non-naturally occurring sequence. A non-naturally occurring functional variant may be a modified version of protein SIC having a modified sequence to that set out as the amino acid sequence of SEQ ID NO: 1, obtained by, for example, amino acid substitution, deletion or addition. Up to 1, up to 5, up to 10, up to 50 or up to 100 amino acid substitutions or deletions, for example, may be made. Thus, a functional variant of the protein SIC may be a fragment of that sequence. Typically, if substitutions are made, the substitutions will be conservative substitutions, for example according to the following Table. Amino acids in the same block in the second column and preferably in the same line in the third column may be substituted for each other. Deletions are preferably deletions of amino acids from one or both ends of the sequence of protein SIC or of deletions of one or more of the repeat regions of protein SIC. Alternatively, deletions are of regions not involved in the interaction or inactivation of antibacterial peptides.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Typically, protein SIC is provided having been produced in recombinant form from a suitable host cell. Alternatively, protein SIC can be provided by providing bacterial cells that express protein SIC endogenously or through the use of recombinant techniques. For example, the assays of the invention may use a suitable strain of S. pyogenes or other bacteria which expresses protein SIC. Such bacteria are incubated with an antibacterial peptide in the presence of the test substance. Alternatively, a separate source of protein SIC can be provided which has either been produced by recombinant or other suitable techniques or alternatively has been isolated from protein SIC expressing bacterial strains.

Protein SIC or a functional variant thereof may be fused to a carrier polypeptide. Thus, additional amino acid residues may be provided at, for example, one or both termini of protein SIC or a functional variant thereof for the purpose of providing a carrier polypeptide, by which the polypeptide can be, for example, affixed to a label, solid matrix or carrier. Thus the first component for use in a method of the invention may be in the form of a fusion polypeptide which comprises heterologous sequences. Typically, fusion polypeptides will comprise a polypeptide sequence as described above and a carrier or linker sequence.

Polypeptides may be modified by, for example, addition of histidine residues, a T7 tag or glutathione S-transferase, to assist in their isolation. Alternatively, the carrier polypeptide may, for example, promote secretion of the polypeptide from a cell or target expression of the polypeptide to the cell membrane. Amino acids carriers can be from 1 to 400 amino acids in length or more typically from 5 to 200 residues in length. The polypeptide may be linked to a carrier polypeptide directly or via an intervening linker sequence. Typical amino acid residues used for linking are tyrosine, cysteine, lysine, glutamic acid or aspartic acid.

Suitable polypeptides for use as a first component may be chemically modified, for example, post translationally modified. For example they may be glycosylated or comprise modified amino acid residues. Polypeptides can be in a variety of forms of polypeptide derivatives, including amides and conjugates with polypeptides.

Chemically modified polypeptides also include those having one or more residues chemically derivatized by reaction of a functional side group. Such derivatized side groups include those which have been derivatized to form amine hydrochlorides, p-toluene sulfonyl groups, carbobenzoxy groups, t-butyloxycarbonyl groups, chloroacetyl groups and formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

Also included as chemically modified polypeptides are those polypeptides which contain one or more naturally occurring amino acids derivatives of the twenty standard amino acids. For example, 4-hydroxyproline may be substituted for proline or homoserine may be substituted for serine.

Protein SIC or a functional variant thereof and/or other polypeptides used as part of a first component may carry a revealing label. Suitable labels include radioisotopes such as ³²P or ³⁵S, fluorescent labels, enzyme labels, or other protein labels such as biotin.

Protein SIC or a functional variant thereof and/or other polypeptides used as part of a first component may be expressed using recombinant DNA techniques. For example, suitable polypeptides may be expressed in, for example, bacterial or insect cell lines (see, for example, Munger et al., 1998, Molecular Biology of the Cell, 9, 2627-2638). Such polypeptides are produced by providing a polynucleotide encoding protein SIC, such as a polynucleotide of SEQ ID NO: 2. Such polynucleotides are provided with suitable control elements, such as promoter sequences, and provided in expression vectors and the like for expression of protein SIC. Also, suitable polypeptides may be isolated biochemically from any suitable bacteria.

Alternatively, polypeptides may be chemically synthesized. Synthetic techniques, such as a solid-phase Merrifield-type synthesis, may be preferred for reasons of purity, antigenic specificity, freedom from unwanted side products and ease of production. Suitable techniques for solid-phase peptide synthesis are well known to those skilled in the art (see for example, Merrifield et al., 1969, Adv. Enzymol 32, 221-96 and Fields et al., 1990, Int. J. Peptide Protein Res, 35, 161-214). In general, solid-phase synthesis methods comprise the sequential addition of one or more amino acid residues or suitably protected amino acid residues to a growing peptide chain.

The second component comprises an antibacterial peptide. A number of antibacterial peptides have been identified and characterised including amongst others human neutrophil α-defensin and LL37. The second component may also comprise a functional variant of a naturally occurring protein, in particular which retains the antibacterial activity of the peptide. The sequences of α-defensin and or LL-37 are set out in SEQ ID NO: 3 and SEQ ID NO: 4. These proteins may be produced by any suitable technique including recombinant techniques and chemical synthesis as described in more detail for the first component polypeptide. Functional variants may have variations such as substitutions or deletions, or sequence identity to SEQ ID NO: 3 or 4 as described above for protein SIC.

The assay can be carried out according to any suitable protocol. Preferably, the assay is adapted so that it can be carried out in a single reaction vessel more preferably can be carried out in a single well of a plastics Microtiter plate and thus can be adapted for high throughput screening.

The assays of the present invention monitor the interaction or activity between protein SIC and an antibacterial peptide. The interaction can be detected in a simple binding assay, for example by coating one of the components to a solid support and monitoring for binding of the second component, for example, using a suitable label such as a radioactive label or fluorescent label on the component. Alternative assays of the invention monitor for the activity of protein SIC on the antibacterial peptides. For example, the two components may be incubated together in the presence of a test substance and the extent, if any, of the reduction in activity of the antibacterial peptide can be monitored.

In an example of a suitable assay format, the antibacterial activity of the antibacterial peptides can be monitored. This activity can be monitored by any suitable means. The activity of the antibacterial peptide can be determined after the peptide has been incubated with a test substance and protein SIC, for example, by monitoring for the antibacterial effect of the antibacterial peptide in the presence of bacteria. Assays can be performed in which bacteria are also present in the assay sample during the course of the assay and the growth of the bacteria can be monitored during the course of the assay. Alternatively, after incubation of protein SIC and antibacterial peptide in the presence of the test substance, the assay sample can be contacted with bacteria in a separate vessel. The assays of activity or degradation of antibacterial peptides can be carried out at selected time points following addition of the test substance to determine the cause of the reaction. The components may be added together in any order, although typically protein SIC and the antibacterial peptide are only incubated together in the presence of the test substance.

In one embodiment, protein SIC is provided by S. pyogenes or other recombinant bacteria which express protein SIC and the activity of the antibacterial peptides is monitored by monitoring the growth of those bacteria, in the presence of a test substance. Assays may be carried out in which additional amounts of an antibacterial peptide are added to the sample to establish the amount of antibacterial peptide that is required to be added to obtain an antibacterial effect, to thereby determine the activity of initial quantity of antibacterial peptides. Such an assay can thus establish the effect of the test substance on the activity of protein SIC on the antibacterial peptide.

Suitable control experiments may be carried out. For example, assays may be carried out in the absence of a test substance to monitor for the activity of protein SIC on the antibacterial peptides in the absence of a test substance. Assays may also be carried out in the absence of antibacterial peptides, to distinguish between agents which inhibit bacterial growth generally, rather than having a specific activity on the protein SIC activity on antibacterial peptides.

The invention also provides a test kit for the identification of a modulator of the interaction or activity of protein SIC on antibacterial peptide. A kit according to the invention comprises a first component and a second component both as described above. A preferred kit of the invention will also comprise means for determining whether a test substance modulates or alters the interaction or activity of protein SIC on the antibacterial peptide. A kit of the invention may optionally further comprise a appropriate buffer(s) controls and may also comprise appropriate packaging and instructions for use in a method of the invention.

Suitable test products which can be tested in the above assays include combinatorial libraries, defined chemical entities, peptide and peptide mimetics, oligonucleotides and natural product libraries, such as display (e.g. phage display libraries) and antibody products. Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, 10 substances per reaction, and the substances of these batches which show inihibition or stimulation tested individually. Test substances may be used at a concentration of from 1 nM to 1000 μM, preferably from 1 μM to 100 μM, more preferably from 1 μM to 10 μM.

An inhibitor of protein SIC activity/interaction with antibacterial peptide is one which produces a measurable reduction on the activity or interaction in the assay as described above. An inhibitor of the interaction between protein SIC and an antibacterial peptide is one which causes the degree of interaction and in particular the activity of protein SIC on antibacterial peptide to be reduced or substantially eliminated, as compared to the degree of interaction between the two, in the absence of that inhibitor. Preferred inhibitors are those which inhibit the interaction/activity between protein SIC and the antibacterial peptide by at least 10%, at least 20%, at least 30%, at least 40%, at least 50%, at least 60%, at least 70%, at least 80%, at least 90%, at least 95% or at least 99% at a concentration of the inhibitor of 1 μgml⁻¹, 10 μgml⁻¹, 100 μgml⁻¹, 500 μgml⁻¹, 1 mgml⁻¹, 10 mgml⁻¹, 100 mg ml⁻¹. The percentage inhibition represents the percentage decrease in the interaction between protein SIC and the antibacterial peptide in a comparison of assays in the presence and absence of the test substance. Any combination of the above mentioned degrees of percentage inhibition and concentration of inhibitor may be used to define an inhibitor of the invention, with greater inhibition at lower concentrations being preferred.

Modulators of the invention may be in substantially purified form. They may be in substantially isolated form, in which case they will generally comprise at least 80% e.g. at least 90, 95, 97 or 99% by weight of the dry mass in the preparation. The product is typically substantially free of other cellular components. The product may be used in such a substantially isolated, purified or free form in the method or be present in such forms in a kit.

Modulators of the invention may be used in a method of treatment of the human or animal body by therapy.

In particular, inhibitors of the invention may be used in the treatment of bacterial infections and in particular in the treatment of bacterial infections by S. pyogenes. The inhibitors may be used alone or in combination with antibacterial peptides such as α-defensin and/or LL-37. Such modulators may be used in prophylactic treatment, for example, in immunosuppressed patients more susceptible to infection by S. pyogenes. Alternatively, such agents may be used in patients demonstrated to have S. pyogenes infections. A therapeutically effective amount of inhibitor may be given to a host in need thereof.

Modulators of protein SIC activity may be administered in a variety of dosage forms. Thus, they can be administered orally, for example as tablets, troches, lozenges, aqueous or oily suspensions, dispersible powders or granules. The modulators may also be administered parenterally, either subcutaneously, intravenously, intramuscularly, intrasternally, transdermally or by infusion techniques. The modulators may also be administered as suppositories. A physician will be able to determine the required route of administration for each particular patient.

The formulation of a modulator for use in preventing or treating bacterial infection will depend upon factors such as the nature of the exact modulator, whether a pharmaceutical or veterinary use is intended, etc. A modulator may be formulated for simultaneous, separate or sequential use.

A modulator is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate; glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of a modulator is administered to a patient. The dose of a modulator may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

### EXAMPLES

Bacterial strains, and purification of protein SIC—The S. pyogenes strains AP1 (40/58) of serotype M1 and AP12 (1/71) of serotype M12 were from the WHO Collaborating Centre for Reference and Research on Streptococci, Prague, Czech Republic. The S. pyogenes strain W38 (GT 71-154) of serotype M55 was from the late Dr. L. W. Wannamaker. Bacteria were grown in Todd-Hewitt broth (TH; Difco, Detroit, Mich.) at 37° C. Protein SIC was purified from the S. pyogenes strains AP1, AP12 and W38 as described (1) by precipitation of the culture medium with 30% ammonium sulfate, followed by ion-exchange chromatography on Mono Q (Pharmacia-Biotech, Uppsala, Sweden). Fractions containing protein SIC were combined, dialysed against 2 mM NH₄HCO₃ and freeze-dried. For the antimicrobial assay (see below) protein SIC was dissolved in 10 mM Tris-HCl, pH 7.5, containing 5 mM glucose.

Proteins, peptides, antibodies and radiolabelling—Recombinant M1 protein was prepared as described previously (10), α-defensin (HNP-1), ACYCRIPACIAGERRYGTCIYQGRLWAFCC (Mw 3442) was purchased from Sigma, and LL-37, LLGDFFRKSKEKIGKEFKRIVQRIKDFLRNLVPRTES (Mw 4492) was synthesized by Innovagen AB, Lund, Sweden. The peptide GCP28, GCPRDIPTNSPELEETLTHTITKLNAEN, based on a sequence in human kininogen has been described previously (11) and was a kind gift from Dr. Heiko Herwald, Lund University, Lund, Sweden Mouse monoclonal α-defensin antibodies were from Bachem AG, and rabbit polyclonal LL-37 antibodies were from Innovagen AB. Antisera against protein SIC was raised in rabbits. Protein SIC was labelled with ¹²⁵I using the chloramine T method (12).

Antimicrobial assay—AP1 bacteria were grown to mid-log phase in TH broth, washed and diluted in 10 mM Tris-HCl, pH 7.5, containing 5 mM glucose. 50 μl bacteria (2×10⁶ colony forming units (cfu)/ml) were incubated together with α-defensin or LL-37 at various concentrations for 2 h at 37° C. In subsequent experiments, bacteria were incubated with α-defensin and LL-37 at a concentration of 448 nM together with different concentrations of protein SIC or M1 protein and the reactions were carried out for 2 h (α-defensin) or 1 h (LL-37). To quantitate the bactericidal activity serial dilutions of the incubation mixtures were plated on TH agar, incubated overnight at 37° C., and the number of cfus were determined.

Bacterial growth assay—AP1 bacteria were grown to stationary phase in TH broth. 200 μl of TH was inoculated with 5 μl of the bacterial suspension in 96-well plates (Falcon) at 37° C. At early logarithmic phase various amounts of LL-37 was added and growth was followed by measuring the absorbance at 490 nm (using a BioRad 550 microplate reader). The amount of protein SIC in the growth medium was estimated by ELISA (see below).

Slot-binding and SDS-polyacrylamide gel electrophoresis—Peptides were applied to polyvinylidene difluoride (PVDF) membranes (Immobilon, Millipore) using a Milliblot-D system (Millipore). Membranes were blocked in TBS (0.05 M Tris-HCl, pH 7.5, 0.15 M NaCl) containing 3% bovine serum albumin (SA), incubated with ¹²⁵I-labelled protein SIC for 3 h, and washed with TBS containing 0.05% Tween-20. Autoradiography was carried out using Kodak x-Omat AR films and regular intesifying screens. SDS-polyacrylamide gel electrophoresis (PAGE) was performed as described by Laemmli (13) using a polyacrylamide concentration of 10% and 3.3% cross-liinkng. Samples were boiled for 3 min in sample buffer containing 2% SDS and 5% 2-mercaptoethanol. Gels were stained with Coomassie blue.

ELISA—Indirect ELISA was performed by coating microtiter plates (Maxisorb, NUNC, Denmark) over night with proteins in serial dilutions (starting at 58 nM). The plates were washed in PBS containing 0.05% Tween-20 (PBST), blocked in PBST containing 2% BSA for 30 min and incubated with 580 nM α-defensin or LL-37 for 1 h. Bound antibacterial peptides were detected with specific antbodies against α-defensin (1:2000) or LL-37 (1:5000), and binding was visualized by a horseradish peroxidase-conjugated secondary antibody against mouse or rabbit IgG (1:3000). All incubations were performed at 37° C. for 1 h followed by a washing step. Substrate solution, 0.1% (w/v) diammonium-2,2-azino-bis-(3-ethyl-2,3-dihydrobenzthiazoline)-6-sulfonate (ABTS), 0.012% (v/v) H₂O₂ in 100 mM NaH₂PO₄, pH4.5, was added and the change in absorbance at 405 nm was determined after 5 min. To determine the concentration of SIC in growth medium plates coated with AP1 growth medium were incubated with antibodies against protein SIC (1:1000) followed by a secondary antibody against rabbit IgG (1:3000). Visualization of binding was detected as above and absorbance was determined after 30 min.

Results

Two major and well-characterized human antibacterial peptides, α-defensin (HNP-1) and LL-37, were used in this investigation. These peptides have broad antibacterial activity against both Gram-positive and Gram-negative bacteria. α-defensin (HNP-1) is found in the azurophilic granules of human neutrophils. Analogues to neutrophil α-defensins are produced also by intestinal Paneth cells (14, 15). LL-37 is produced by neutrophils and epithelial cells. α-defensin and LL-37 are both found in extracellular fluids, including wound fluid, and the two peptides act synergistically on target bacteria (16).

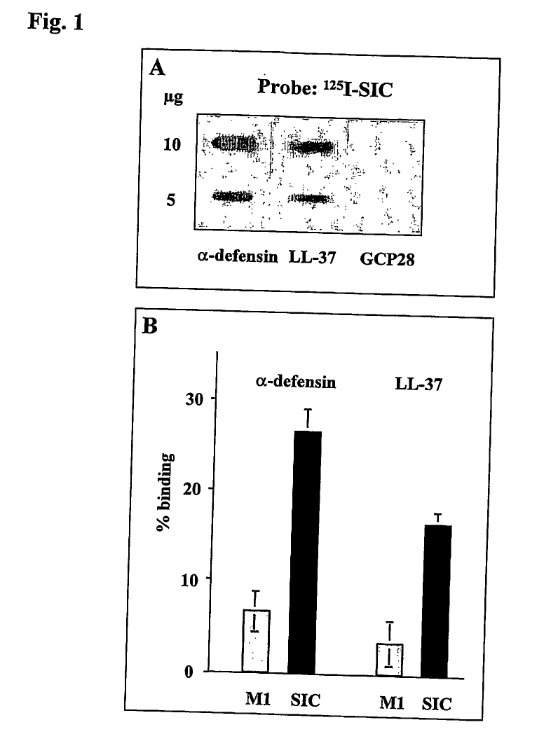

In S. pyogenes, the sic gene is part of the so-called mga regulon. Like the other genes of the mga regulon, sic is expressed at an early growth phase (1), suggesting that SIC will be secreted as soon as S. pyogenes bacteria carrying the sic gene starts to grow on an epithelial surface. To investigate whether α-defensin and LL-37 have affinity for SIC, these peptides and a control peptide (GCP28) based on a sequence in H-kininogen (11), were applied to Immobilon filters which were probed with ¹²⁵I-labelled SIC (if not indicated otherwise SIC is from the M1 strain AP1). FIG. 1A shows that SIC interacts with the antibacterial peptides, an observation which was confirmed also by experiments where SIC and M1 protein were applied to plastic wells, followed by the addition of α-defensin or LL-37 and antibodies to the peptides. M1 protein was chosen as a control. It was isolated from the same strain of S. pyogenes (AP1) as SIC (1), and although the protein is predominantly associated with the cell wall, it is also released from the bacterial surface by proteolytic cleavage (17). In the experiments summarized in FIG. 1B, α-defensin and LL-37 showed affinity for SIC, whereas the interaction with M1 protein was at background level.

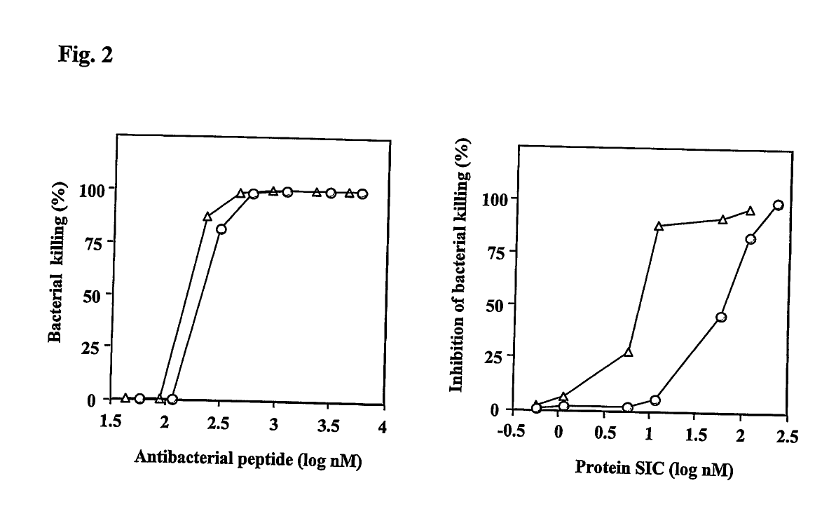

Next we investigated the antibacterial effect of α-defensin and LL-37 on the AP1 strain. In these experiments the bacteria were washed and resuspended in buffer prior to the addition of the peptides to exclude that SIC was present during the incubation period. As shown in the left panel of FIG. 2, the peptides killed the bacteria. The concentration required for 100% killing was approximately 0.4 μM for both peptides. The inhibitory effect of SIC was then tested at a bactericidal concentration (0.448 μM) of the peptides and the results (see right panel, FIG. 2) show that SIC blocks the antibacterial activity of α-defensin and LL-37. The inhibition curves indicate that SIC is about ten times more efficient in blocking LL-37 than α-defensin. M1 protein was also tested (at a maximum of 0.72 μM) but showed no inhibitory activity.

When grown to stationary phase, the growth medium of S. pyogenes of the M1 serotype contains large quantities (10-15 μg/ml) of protein SIC (1) and in a series of experiments we investigated whether SIC produced by growing M1 bacteria, could protect the organisms against LL-37 (these experiments required substantial amounts of antibacterial peptide which is why α-defensin was not tested). The AP1 strain was grown to early logarithmic growth phase where the concentration of SIC was 4-5 μg/ml growth medium as determined by ELISA. At this point different amounts of LL-37 were added. Compared to the experiments where the bacteria were washed to remove SIC before the addition of LL-37 (see FIG. 2), the concentration of LL-37 required to kill 50% of the bacteria, was more than 50 times higher (11.1 μM compared to 0.2 μM). The results suggest that SIC secretion provides effective protection against antibacterial peptides already at an initial stage of infection.

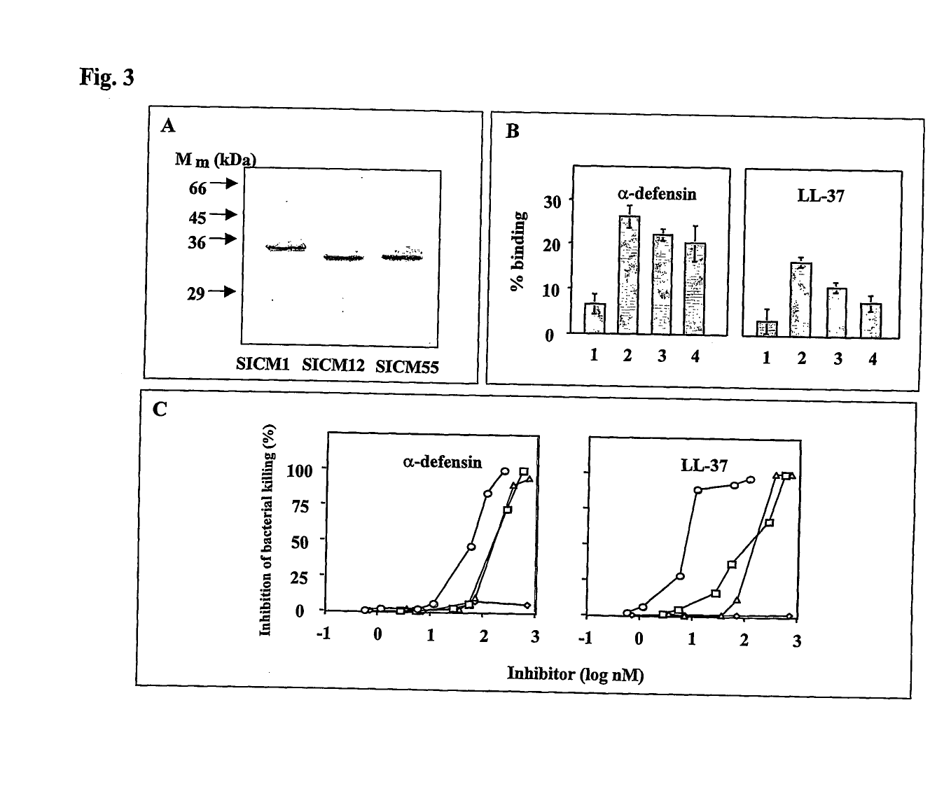

As mentioned, SIC homologues are produced by S. pyogenes strains of M serotypes 1, 12, 55, and 57 (1, 2). Using the same isolation protocol as for SIC from M1 bacteria (1), SIC was purified from the growth medium of M12 and M55 organisms. Analogous to the M1 strain (1), the M12 and M55 strains produced 10-15 μg SIC/ml growth medium when grown to stationary phase. The purified homologues (FIG. 3A) and M1 protein were then added to microtiter plates and the binding of α-defensin and LL-37 was determined as described above (see FIG. 1B). α-defensin interacted with the three SIC proteins, whereas in this experimental system only SICM1 showed binding of LL-37 clearly above M1 protein, the negative control (FIG. 3B). When tested for their ability to interfere with the antibacterial activity of α-defensin and LL-37, all SIC homologues, but not M1 protein, blocked this activity (FIG. 3C). The inhibition curves show that SICM1 is 5-10 times more potent in this-respect than the M12 and M55 homologues. It is noteworthy that the four SIC-producing M serotypes (1, 12, 55, and 57) are all known to be associated with poststreptococcal glomerulonephritis, suggesting a role for SIC in this condition (1, 2). Moreover, the fact that M1 strains dominate in cases of invasive disease and that SICM1 is the most potent inhibitor of α-defensin and LL-37, support the notion that SIC interference with antibacterial peptides facilitates S. pyogenes invasion through mucosal and skin barriers.

Research in many laboratories has over the past 10-20 years firmly established the fundamental role played by antibacterial peptides in the initial clearance of pathogenic bacteria. The demonstration that SIC not only interferes with complement function, but also inactivates antibacterial peptides, further underlines the significance of the innate immune system. The data of the present study also emphasize the highly complex molecular interplay between S. pyogenes and its human host. Although pathogenicity and virulence are polygenic properties, the results indicate that SIC could represent an important virulence determinant. A previous investigation (18) showed that an isogenic M1 mutant strain in which the sic gene had been inactivated, was significantly less efficient in colonizing the throat of mice as compared to the wild-type strain. These data demonstrate that SIC promotes the early stages of infection and this study suggests that inactivation of antibacterial peptides could be the molecular mechanism behind this effect. The results of the present work may also explain the unique variability of the sic gene and the fact that the M1 serotype is the serotype most frequently connected with invasive S. pyogenes infection.

### REFERENCES

1. Åkesson, P., Sjöholm, A. G., and Björck, L. (1996) J. Biol,. Chem. 271, 1081-1088.
2. Hartas, J., and Sriprakash, K. S. (1999) Microb Pathog 26, 25-33.
3. Fernie-King, B. A., Seilly, D. J., Willers, C., Wurzner, R., Davies, A., and Lachmann, P. J. (2001) Immunology 103, 390-8.
4. Stockbauer, K. E., Grigsby, D., Pan, X., Fu, Y. X., Mejia, L. M., Cravioto, A., and Musser, J. M. (1998) Proc Natl Acad Sci USA 95, 3128-33.
5. Hoe, N. P., Nakashima, K., Lukomski, S., Grigsby, D., Liu, M., Kordari, P., Dou, S. J., Pan, X., Vuopio-Varkila, J., Salmelinna, S., McGeer, A., Low, D. E., Schwartz, B., Schuchat, A., Naidich, S., De Lorenzo, D., Fu, Y. X., and Musser, J. M. (1999) Nat med 5, 924-9.
6. Boman, H. G. (2000) Immunol. Rev. 173, 5-16.
7. Lehrer, R. I., and Ganz, T. (1999) Curr. Opin. Immunol. 11, 23-27.
8. Schröder, J.-M., and Harder, J. (1999) Int J Biochem Cell Biol 31, 645-651.
9. Selsted, M. E., and Ouellette, A. J. (1995) Trends cell Biol 5, 114-119.
10. Åkesson, P., Schmidt, K.-H., Cooney, J., and Björck, L. (1994) Biochem. J. 300, 877-886.
11. Herwald, H., Hasan, A. A. K., J., G.-Z., Schmaier, A. H., and Müller-Esterl, W. (1995) J. Biol. Chem. 270, 14634-14642.
12. Greenwood, F. C., Hunter, W. M., and Glover, J. S. (1963) Biochem. J. 89, 114-123.
13. Laemmli, U. K. (1970) Nature 227, 680-685.
14. Ouellette, A. J., and Selsted, M. E. (1996) FASEB J 10, 1280-1289.
15. Ayabe, T., Satchell, D. P., Pesendorfer, P., Tanabe, H., Wilson, C. L., Hagen, S. J., and Ouellette, A. J. (2002) J. Biol Chem 227, 5219-28.
16. Nagaoka, I., Hirota, S., Yomogida, S., Ohwada, A., and Hirata, M. (2000) Inflamm Res 49, 73-9.
17. Berge, A., and Björck, L. (1995) J. Biol. Chem. 270, 9862-9867.
18. Lukomski, S., Hoe, N. P., Abdi, I., Rurangirwa, J., Kordari, P., Liu, M., Dou, S. J., Adams, G. G., and Musser, J. M. (2000) Infect Immun 68, 535-42.

1. An assay method for an anti-bacterial agent comprising:
(a) providing as a first component protein SIC;
(b) providing as a second component an antibacterial peptide;
(c) contacting the first component with a test substance in the presence of the second component; and
(d) determining the interaction or activity of the first component with the second component to determine thereby whether a test substance is an effective anti-bacterial agent.

2. A method according to claim 1 wherein the first component protein SIC comprises wild type protein SIC derived from S. pyogenes, a fragment thereof which maintains the ability to inactivate human neutrophil α-defensin and LL-37, or a functional variant of either thereof which maintains the ability to inactivate human neutrophil α-defensin or LL-37.

3. A method according to claim 2 wherein protein SIC is derived from S. pyogenes of serotype selected from M1, M12, M57 or M55.

4. A method according to claim 2 wherein protein SIC is derived from S. pyogenes of M1 serotype.

5. A method according to claim 4 wherein protein SIC has the sequence of SEQ ID NO: 1.

6. A method according to claim 1 wherein the anti-bacterial peptide is selected from α-defensin, LL-37 or a mixture thereof.

7. A method according to claim 1 wherein step (d) comprises monitoring the interaction of the antibacterial peptide with protein SIC.

8. A method according to claim 1 wherein step (d) comprises monitoring the activity of the antibacterial peptide.

9. A method according to claim 8 wherein step (d) comprises contacting the assay sample with bacteria to determine whether the antibacterial peptide has retained antibacterial activity.

10. A method according to claim 9 wherein step (a) comprises providing bacteria which express protein SIC and wherein step (d) comprises monitoring the growth of the bacteria in the presence of a test substance.

11. A method according to claim 9 wherein the method comprises determining the amount of an additional antibacterial peptide which is required to confer antibacterial activity in the presence of the test substance, to thereby determine the residual activity of the antibacterial peptide provided in step (b).

12. A method according to claim 1 further comprising the step of formulating an agent identified as an inhibitor of protein SIC activity as a pharmaceutical composition with a pharmaceutically acceptable carrier.

13. (canceled)

14. (canceled)

15. A method of treating an individual suffering from S. pyogenes infection comprising administering an inhibitor of protein SIC.

16. A pharmaceutical composition comprising an inhibitor of protein SIC identifiable by a method of claim 1 and a pharmaceutically acceptable carrier.

17. A method according to claim 15 wherein said inhibitor is administered together with an antibacterial peptide.


SEQUENCE LISTING (5 sequences)

SEQ ID NO: 1 - protein, 305 aa, Streptococcus pyogenes
MNIRNKIENSKTLLFTSLVAVALLGATQPVSAETYTSRNFDWSGDDWPEDDWSGDGLSKYDRSGVGLSQYGWSQYGWSSDKEEWPEDWPEDDWSSDKKDETEDKTRPPYGEALGTGYEKRDDWGGPGTVATDPYTPPYGGALGTGYEKRDDWGGPGTVAIDPYTPPYGEALGTGYEKRDDWRGPGHIPKPENEQSPNPSHIPEPPQIEWPQWNGFDELSFGPSDWGQSEDAPRFPSEPRVPEKPQHTPQKNPQESDFDRGFSAGLKAKNSGRGIDFEGFQYGGWSDEYKKGYMQAFGTPYTPSAT

SEQ ID NO: 2 - DNA, 915 bp, Streptococcus pyogenes
atgaatactagaaataagattgaaaatagtaaaacactactatttacatcccttgtagccgtggctctactaggagctacacaaccagtttcagccgaaacgtatacatcacgcaattttgactggtctggagatgactggcctgaagatgactggtctggagatggtttgtctaaatatgaccggtctggagttggtttgtctcaatatggctggtctcaatatggctggtctagcgataaagaagaatggcctgaagattggcctgaagatgactggtctagcgataaaaaagatgagacagaagataaaacgagaccaccatatggagaagcattaggtacagggtatgaaaaacgtgatgattggggaggacctggtacggtggcaactgacccttacactccaccatatggaggagcattaggtacagggtatgaaaaacgtgatgattggggaggacctggtacggtagcaattgacccttacactccaccatatggagaagcattaggtacagggtatgaaaaacgtgatgattggagaggacctggacatattcctaaacctgagaacgaacaatcaccaaacccatctcatattcctgaacctcctcagattgagtggcctcagtggaatggctttgatgaattatcatttggcccctctgattggggccaatctgaggacgcccctcgtttcccaagtgaacctcgtgtgccagaaaaaccgcaacatactcctcaaaaaaatccacaagaatcagattttgatagagggttttcagctggcttgaaagcaaaaaactcaggtagaggtattgattttgaaggtttccagtatggtggctggtcagacgaatataaaaaaggttacatgcaagccttcggtacaccatatacaccatcagcaacg

SEQ ID NO: 3 - protein, 30 aa, Homo sapiens
ACYCRIPACIAGERRYGTCIYQGRLWAFCC

SEQ ID NO: 4 - protein, 37 aa, Homo sapiens
LLGDFFRKSKEKIGKEFKRIVQRIKDFLRNLVPRTES

SEQ ID NO: 5 - protein, 28 aa, Homo sapiens
GCPRDIPTNSPELEETLTHTITKLNAEN